(19) United States
(12) Patent Application Publication
(10) Pub. No.: US 2024/0390486 A1
(43) Pub. Date: Nov. 28, 2024

(54) PEANUT ALLERGY EPITOPE

(71) Applicant: Vanderbilt University, Nashville, TN (US)

(72) Inventors: Benjamin W. Spiller, Nashville, TN (US); Scott A. Smith, Nashville, TN (US)

(21) Appl. No.: 18/695,945

(22) PCT Filed: Oct. 6, 2022
(86) PCT No.: PCT/US2022/077638

§ 371 Date: Mar. 27, 2024

Related U.S. Application Data
(60) Provisional application No. 63/252,710, filed on Oct. 6, 2021.

(51) Int. Cl. A61K 39/35; A61P 37/08; G01N 33/68

(52) U.S. Cl. CPC A61K 39/35; A61P 37/08; G01N 33/6854; G01N 2333/415; G01N 2800/52

(57) ABSTRACT

Disclosed herein is a composition comprising a mutant peanut (Arachis hypogaea) Ara h 6 polypeptide, a mutant Ara h 2 polypeptide, or a combination thereof in a pharmaceutically acceptable carrier, wherein the mutant Ara h 6 polypeptide and/or mutant Ara h 2 polypeptide has a reduced IgE response in a subject with a peanut allergy compared to wildtype Ara h 6 or Ara h 2 polypeptide. Also disclosed is a method for desensitizing or tolerizing a subject with a peanut allergy, comprising administering to the subject the mutant peanut Ara h 6 and/or Ara h 2 polypeptides disclosed herein. Also disclosed is a method for monitoring progress for the disclosed therapeutic methods.

### CROSS-REFERENCE TO RELATED APPLICATIONS

This application claims benefit of U.S. Provisional Application No. 63/252,710, filed Oct. 6, 2021, which is hereby incorporated herein by reference in its entirety.

### STATEMENT REGARDING FEDERALLY SPONSORED RESEARCH OR DEVELOPMENT

This invention was made with Government Support under Grant No. A1155668 awarded by the National Institutes of Health. The Government has certain rights in the invention.

### SEQUENCE LISTING

This application contains a sequence listing filed in ST.26 format entitled “222230_2120_Sequence_Listing” created on Oct. 4, 2022. The content of the sequence listing is incorporated herein in its entirety.

### BACKGROUND

Peanut allergy is common, affecting 1-2% of young children in Europe and the USA, and unlike other common childhood food allergies, resolution is uncommon. The quality of life of the affected families is reduced because of constant fear over food choices and the likelihood of anaphylaxis. Despite the current best management, families of peanut allergic children have poor knowledge of how to avoid and also treat food allergy emergencies. Accidental reactions are common, and nearly one third of nut-allergic children cannot recognize the nut to which they are allergic, putting them at increased risk of unintentional ingestion. There is therefore a need to develop a disease-modifying therapy for peanut allergy.

### SUMMARY

Peanuts (Arachis hypogaea) contain multiple allergenic proteins, including Ara h 1 to Ara h 9. An individual with a peanut allergy may be hypersensitive to one or more of these allergenic peanut proteins. Patients who are hypersensitive to any peanut allergen or combination of peanut allergens may be treated using the methods described herein.

Most individuals with peanut allergy from Western European populations are typically display an allergic response to Ara h 2. A patient with a peanut allergy may display peanut-specific serum IgE, i.e. IgE which specifically binds to peanut protein.

Patients may be diagnosed with peanut allergy according to standard clinical criteria. Standard clinical criteria may include for example, a history of a type-1 hypersensitivity reaction which is temporally related to peanut ingestion (e.g. hives, swelling, wheezing, abdominal pain, vomiting, breathlessness), and the presence of peanut-specific IgE by positive skin prick test (wheal diameter >/=3 mm) or ImmunoCap serum IgE >0.35 kU/1.

In some embodiments, the methods described herein may be used for any patient with peanut allergy and are independent of the patient's sensitivity or challenge threshold to peanut allergen, the weight or height of the patient and other factors.

Disclosed herein are crystal structures of peanut allergens (Ara h 2 and Ara h 6) bound to IgE antibodies derived from human subjects. Four dominant epitopes on the Ara proteins have been discovered. In some embodiments, these disclosed epitopes can be used to develop diagnostic and therapeutic agents.

Therefore, disclosed herein is a composition comprising a mutant peanut (Arachis hypogaea) Ara h 6 polypeptide, a mutant Ara h 2 polypeptide, or a combination thereof in a pharmaceutically acceptable carrier, wherein the mutant Ara h 6 polypeptide and/or mutant Ara h 2 polypeptide has a reduced IgE response in a subject with a peanut allergy compared to wildtype Ara h 6 or Ara h 2 polypeptide.

Also disclosed is a method for desensitizing or tolerizing a subject with a peanut allergy, comprising administering to the subject the mutant peanut Ara h 6 and/or Ara h 2 polypeptides disclosed herein.

Also disclosed is a method for monitoring progress for the disclosed therapeutic methods that involves assaying a sample from the subject for IgE and/or IgG antibodies that selectively bind one or more of the disclosed mutant peanut polypeptides, wherein a reduction in IgE antibodies and/or an increase in IgG antibodies that selectively bind the one or more mutant peanut polypeptides is an indication that the subject is being desensitized or tolerized to the peanut allergy.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1: Site A is defined by the residues on Ara h 6 that are recognized by a site A targeted antibody (16A8) and Ara h 6. Panel A shows an overview of the 16A8-Ara h 6 interaction. Panel B shows a space-filling model with site A on Ara h 6. Panel C shows stick representation of site A. The residues Q113 and R114 abolish binding when mutated.

FIG. 2: Site C is defined by the residues on Ara h 6 that are recognized by a site C targeted antibody (1 H9, shown in gold and green) and Ara h 6. Panel A shows an overview of the 1 H9-Ara h 6 interaction. Panel B shows a space-filling model with site C.

Panel C shows stick representation of site C. The residues R90 and Q91 abolish binding when mutated.

FIG. 3: Site D is defined by the residues on Ara h 6 that are recognized by a site D targeted antibody (8F3) and Ara h 6. Panel A shows an overview of the 1H9-Ara h 6 interaction. Panel B shows a space-filling model with site D on Ara h 6. Panel C shows stick representation of site D. The residues R33, Q56, E99, and Q106 abolish binding when mutated.

FIG. 4: Epitope Disruption. The epitopes recognized by Site A, Site C, and Site D antibodies have been identified and are described above. Mutations in each site dramatically reduce binding to antibodies that recognize each site, but do not disrupt binding to antibodies recognizing other, non-mutated, sites. As disclosed herein, mutations within the sites described above can have application as both diagnostics and therapeutics.

FIG. 5: Site B is defined by the residues on Ara h 2 that are recognized by a site B targeted antibody (38B7) and Ara h 2. Panel A and B show an overview of the 38B7-Ara h 2 interaction. Panel C shows a space filling model with site B on Ara h 2. Panel D shows stick representation of site B. Highlighted residues in D are D42, P43, Y44, S45, P46, S47, Q99, G100, R101, and Q102. Site B partially involves binding to a linear epitope that is unstructured when not immobilized by a binding partner. This linear portion of the epitope (DPYSPS) is present two times in Ara h 2.01 and 3 times in Ara h 2.03. The second proline in this sequence is known to be hydroxylated. A structure of the DPYSPS peptide with P46 hydroxylated was determined and reveals that hydroxyl group is recognized by D32 of 38B7 (FIG. 5C).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

The term “non-conservative amino acid substitution” refers to a substitution that is not a conservative amino acid substitution, e.g. having dissimilar side chains. In some embodiments, the non-conservative amino acid substitution involves a change in charge, polarity, hydrophobicity, and/or size.

The terms “peptide,” “protein,” and “polypeptide” are used interchangeably to refer to a natural or synthetic molecule comprising two or more amino acids linked by the carboxyl group of one amino acid to the alpha amino group of another. In addition, as used herein, the term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-E-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

The term “variant” refers to an amino acid or peptide sequence having conservative amino acid substitutions or non-conservative amino acid substitutions with at least 60%, 70%, 80%, 90%, or 95% sequence identity to a reference sequence.

The term “percent (%) sequence identity” or “homology” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “residue” as used herein refers to an amino acid that is incorporated into a polypeptide. The amino acid may be a naturally occurring amino acid and, unless otherwise limited, may encompass known analogs of natural amino acids that can function in a similar manner as naturally occurring amino, acids.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent” refers to a treatment that forestalls or slows the onset of a disease or condition or reduced the severity of the disease or condition. Thus, if a treatment can treat a disease in a subject having symptoms of the disease, it can also prevent that disease in a subject who has yet to suffer some or all of the symptoms.

The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

As used herein, the terms “vaccine therapy”, “vaccination” and “vaccination therapy,” as used interchangeably herein, refer in general to any method resulting in immunological prophylaxis. In one aspect, vaccine therapy induces an immune response, and thus long-acting immunity, to a specific antigen. These methods generally entail the delivery to a subject of an immunogenic material to induce immunity. In an aspect, the “vaccine therapy” refers to a method for the down-regulation of an immune potential to a particular antigen (e.g., to suppress an allergic response). This type of vaccine therapy is also referred to as “tolerance therapy.” Vaccine therapies typically entail a series of parenteral or oral administrations of the immunogenic material over an extended period of time.

### Mutant Antigens

Disclosed herein are mutant peanut (Arachis hypogaea) Ara h 6 and Ara h 2 polypeptide antigens with mutated epitopes that disrupt binding of peanut allergy antibodies. In some embodiments, these antigens can be used as a vaccine to stimulate non-allergy-inducing antibodies (e.g. IgG antibodies) to clear peanut proteins without an allergic response.

In some embodiments, the Ara h 6 wild type protein has the amino acid sequence:

(SEQ ID NO: 1)
MRRERGRQGDSSSCERQVDRVNLKPCEQHIMQRIMGEQEQYDSYDIRST
RSSDQQQRCCDELNEMENTQGCMCEALQQIMENQCDRLQDRQMVQQFKR
ELMSLPQQCNFRAPQRCDLDVSGGRC.

In some embodiments, an alternate Ara h 6 wild type protein has the amino acid sequence:

(SEQ ID NO: 19)
MRRERGRQGDSSSCERQVDRVNLKPCEQHIMQRIMGEQEQYDSYDIRST
RSSDQQQRCCDELNEMENTQGCMCEALQQIMENQCDRLQDRQMVQQFKR
ELRNLPQQCGLRAPQRCDLDVESGGRDRY.

In some embodiments allergy-inducing antibodies bind a first set of contact residues in Ara h 6 (referred to herein as Ara h 6 Site A), as identified by the bold/uppercase residues in the amino acid sequence:
mrrergrqgdssscerqvdrvnikpceqhimqrimgeqeqydsydirstrssdqqqrccdelnemenTQgcmcealq qimenqcdrlqdrqmvqqfkrelMslpqqcnfRAPQRcDIDVSggrc (SEQ ID NO:1) or
mrrergrqgdssscerqvdrvnikpceqhimqrimgeqeqydsydirstrssdqqqrccdelnemenTQgcmcealq qimenqcdrlqdrqmvqqfkrelRnlpqqcglRAPQRcDIDVEsggrdry (SEQ ID NO:19). In some embodiments, any of these bold/uppercase residues can be mutated to disrupt binding by allergy-inducing antibodies. Therefore, in some embodiments, the mutant Ara h 6 antigen comprises the amino acid sequence:
mrrergrqgdssscerqvdrvnikpceqhimqrimgeqeqydsydirstrssdqqqrccdelnemenTQgcmcealq qimenqcdrlqdrqmvqqfkrelMslpqqcnfRAPQRcDIDVSggrc (SEQ ID NO:1), comprising at least 1, 2, 3, 4, 5, 6, 7, 8, 9, 10, 11, 12, 13, or 14 substitutions at amino acid residues T68, Q69, M101, R110, A111, P112, Q113, R114, D116, D118, V119, S120, or a combination thereof. Therefore, in some embodiments, the mutant Ara h 6 antigen comprises the amino acid sequence:
mrrergrqgdssscerqvdrvnikpceqhimqrimgeqeqydsydirstrssdqqqrccdelnemenTQgcmcealq qimenqcdrlqdrqmvqqfkrelRnlpqqcglRAPQRcDIDVeSggrdry (SEQ ID NO:19), comprising at least 1, 2, 3, 4, 5, 6, 7, 8, 9, 10, 11, 12, 13, or 14 substitutions at amino acid residues T68, Q69, R101, R110, A111, P112, Q113, R114, D116, D118, V119, E120, ora combination thereof. In some embodiments, the mutation is a non-conservative mutation, such as a charge-reversal mutation. In some embodiments, mutations of Gln, Glu, and/or Arg residues are preferred. Therefore, in some embodiments, the mutant Ara h 6 antigen comprises the amino acid sequence:
MRRERGRQGDSSSCERQVDRVNLKPCEQHIMQRIMGEQEQYDSYDIRSTRSSDQQQ RCCDELNEMENTQGCMCEALQQIMENQCDRLQDRQMVQQFKRELMSLPQQCNFRA PX₁X₂CDLDVSGGRC (SEQ ID NO:3), wherein X₁ and X₂ are not Gln and Arg, respectively, such as wherein X₁ and X₂ are non-conservative substitutions of Gln and Arg, respectively. For example, in some embodiments, the mutant Ara h 6 antigen comprises the amino acid sequence:

(SEQ ID NO: 11)
MRRERGRQGDSSSCERQVDRVNLKPCEQHIMQRIMGEQEQYDSYDIRST
RSSDQQQRCCDELNEMENTQGCMCEALQQIMENQCDRLQDRQMVQQFKR
ELMSLPQQCNFRAPAECDLDVSGGRCMRRERGRQGDSSSCERQVDRVNL
KPCEQHIMQRIMGEQEQYDSYDIRSTRSSDQQQRCCDELNEMENTQGCM
CEALQQIMENQCDRLQDRQMVQQFKRELMSLPQQCNFRAPAACDLDVSG
GRC.

In some embodiments, allergy-inducing antibodies bind a second set of contact residues in Ara h 6 (referred to herein as Ara h 6 Site B), as identified by the bold/uppercase residues in the amino acid sequence:
mrrergrqgdssscerqvdrvnikpceqhimqrimgeqeqYDsydirstrssdqqqrccdelnementqgcmcealq qimenqcdrlQDRQmvqqfkrelmslpqqcnfrapqrcdldvsggrc (SEQ ID NO:1), or mrrergrqgdssscerqvdrvnikpceqhimqrimgeqeqYDsydirstrssdqqqrccdelnementqgcmcealq qimenqcdrlQDRQmvqqfkrelrnlpqqcglrapqrcdldvesggrdry (SEQ ID NO:19). In some embodiments, any of these bold/uppercase residues can be mutated to disrupt binding by allergy-inducing antibodies. Therefore, in some embodiments, the mutant Ara h 6 antigen comprises the amino acid sequence:
mrrergrqgdssscerqvdrvnikpceqhimqrimgeqeqydsYDsydirstrssdqqqrccdelnementqgcmce alqqimenqcdrlQDRQmvqqfkrelmslpqqcnfrapqrcdldvsggrc (SEQ ID NO:1), comprising at least 1, 2, 3, 4, 5, or 6 substitutions at amino acid residues Y44, D45, Q88, D89, R90, Q91, or a combination thereof. Therefore, in some embodiments, the mutant Ara h 6 antigen comprises the amino acid sequence:
mrrergrqgdssscerqvdrvnikpceqhimqrimgeqeqYDsydirstrssdqqqrccdelnementqgcmcealq qimenqcdrlQDRQmvqqfkrelrnlpqqcglrapqrcdldvesggrdry (SEQ ID NO:19), comprising at least 1, 2, 3, 4, 5, or 6 substitutions at amino acid residues Y41, D42, Q88, D89, R90, Q91, or a combination thereof. In some embodiments, the mutation is a non-conservative mutation, such as a charge-reversal mutation. In some embodiments, mutations of Gln, Glu, and/or Arg residues are preferred. Therefore, in some embodiments, the mutant Ara h 6 antigen comprises the amino acid sequence:
MRRERGRQGDSSSCERQVDRVNLKPCEQHIMQRIMGEQEQYDSydIRSTRSSDQQQR CCDELNEMENTQGCMCEALQQIMENQCDRLqdX₁X₂MVQQFKRELMSLPQQCNFRAP QRCDLDVSGGRC (SEQ ID NO:4), wherein X₁ and X₂ are not Arg and Gln, respectively, such as wherein X₁ and X₂ are non-conservative substitutions of Arg and Gln, respectively. For example, in some embodiments, the mutant Ara h 6 antigen comprises the amino acid sequence:

(SEQ ID NO: 12)
SCERQVDRVNLKPCEQHIMQRIMGEQEQYDSydIRSTRSSDQQQRCCDE
LNEMENTQGCMCEALQQIMENQCDRLqdAAMVQQFKRELMSLPQQCNFR
APQRCDLDVSGGRC.

As disclosed herein, site B involves a long loop that can be deleted. Therefore, in some embodiments, the mutant Ara h 6 antigen comprises a deletion of Q40-R53 and replacement with a short (e.g. GSG or similar) linker in SEQ ID NO:1. Therefore, in some embodiments, the mutant Ara h 6 antigen comprises the amino acid sequence:
Mrrergrqgdssscerqvdrvnikpceqhimqr(GSG)qrccdelnementqgcmcealqqimenqcdrlQDRQm vqqfkrelmslpqqcnfrapqrcdldvsggrc (SEQ ID NO:21).

In some embodiments, allergy-inducing antibodies bind a third set of contact residues in Ara h 6 (referred to herein as Ara h 6 Site C), as identified by the bold/uppercase residues in the amino acid sequence:
mrrergrqgdssscerqvdrvnikpceqhimqrlMgeqeqydsydirstrssdqqqrccdelnementqgcmcealqqi menqCDrLQDRQMVQQfKrelmslpqqcnfrapqrcdlDVsGGRC (SEQ ID NO:1) or
mrrergrqgdssscerqvdrvnikpceqhimqriMgeqeqydsydirstrssdqqqrccdelnementqgcmcealqqi menqCDrLQDRQMVQQfKrelrnlpqqcglrapqrcdlDVesGGRdry (SEQ ID NO:19). In some embodiments, any of these bold/uppercase residues can be mutated to disrupt binding by allergy-inducing antibodies. Therefore, in some embodiments, the mutant Ara h 6 antigen comprises the amino acid sequence:
mrrergrqgdssscerqvdrvnikpceqhimqrlMgeqeqydsydirstrssdqqqrccdelnementqgcmcealqqi menqCDrLQDRQMVQQfKrelmslpqqcnfrapqrcdlDVsGGRC (SEQ ID NO:1), comprising at least 1, 2, 3, 4, 5, 6, 7, 8, 9, 10, 11, 12, 13, 14, 15, 16, 17, or 18 substitutions at amino acid residues M35, C84, D85, L87, Q88, D89, R90, Q91, M92, V93, Q94, Q95, K97, D118, V119, G121, G122, R123, C124, or a combination thereof. Therefore, in some embodiments, the mutant Ara h 6 antigen comprises the amino acid sequence:

(SEQ ID NO: 19)
mrrergrqgdssscerqvdrvnlkpceqhimqriMgeqeqyd
sydirstrssdqqqrccdelnementqgcmcealqqimenqC
DrLQDRQMVQQfKrelrnlpqqcglrapqrcdlDVesGGRdry,

comprising at least 1, 2, 3, 4, 5, 6, 7, 8, 9, 10, 11, 12, 13, 14, 15, 16, 17, or 18 substitutions at amino acid residues M35, C84, D85, L87, Q88, D89, R90, Q91, M92, V93, Q94, Q95, K97, D118, V119, G121, G122, R123, or a combination thereof. In some embodiments, the mutation is a non-conservative mutation, such as a charge-reversal mutation. In some embodiments, mutations of Gln, Glu, and/or Arg residues are preferred. Therefore, in some embodiments, the mutant Ara h 6 antigen comprises the amino acid sequence:
MRRERGRQGDSSSCERQVDRVNLKPCEQHIMQRImGEQEQYDSYDIRSTRSSDQQQ RCCDELNEMENTQGCMCEALQQIMENQcdRIqdX₁X₂mvqqFkRELMSLPQQCNFRAPQ RCDLdvSggrc (SEQ ID NO:5), wherein X₁ and X₂ are not Arg and Gln, respectively, such as wherein X₁ and X₂ are non-conservative substitutions of and Arg and Gln, respectively. For example, in some embodiments, the mutant Ara h 6 antigen comprises the amino acid sequence:

(SEQ ID NO: 13)
MRRERGRQGDSSSCERQVDRVNLKPCEQHIMQRImGEQEQY
DSYDIRSTRSSDQQQRCCDELNEMENTQGCMCEALQQIMEN
QcdRlqdEAmvqqFkRELMSLPQQCNFRAPQRCDLdvSggr
c

In some embodiments, allergy-inducing antibodies bind a fourth set of contact residues in Ara h 6 (referred to herein as Ara h 6 Site D), as identified by the bold/uppercase residues in the amino acid sequence:
mrrergrqgdssscerqvdrvnikpceqHimQRImGeqeqydsydirstrssDqQQrcCdelnementqgcmce alqqimenqcdrlqdrqmvqQfkRElmslpQQCNfrapqrcdldvsggrc (SEQ ID NO:1) or
mrrergrqgdssscerqvdrvnikpceqHimQRImGeqeqydsydirstrssDqQQrcCdelnementqgcmce alqqimenqcdrlqdrqmvqQfkRElrnlpQQCGlrapqrcdldvesggrdry (SEQ ID NO:19). In some embodiments, any of these bold/uppercase residues can be mutated to disrupt binding by allergy-inducing antibodies. Therefore, in some embodiments, the mutant Ara h 6 antigen comprises the amino acid sequence:
mrrergrqgdssscerqvdrvnikpceqHimQRImGeqeqydsydirstrssDqQQrcCdelnementqgcmce alqqimenqcdrlqdrqmvqQfkRElmslpQQCNfrapqrcdldvsggrc (SEQ ID NO:1), comprising at least 1, 2, 3, 4, 5, 6, 7, 8, 9, 10, 11, 12, 13, 14, 15, or 16 substitutions at amino acid residues H29, Q32, R33, 134, G36, D53, Q55, Q56, C59, Q94, R97, E98, Q105, Q106, C107, N108, or a combination thereof. Therefore, in some embodiments, the mutant Ara h 6 antigen comprises the amino acid sequence:

(SEQ ID NO: 19)
mrrergrqgdssscerqvdrvnlkpceqHimQRImGeqeqydsy
dirstrssDqQQrcCdelnementqgcmcealqqimenqcdrlq
drqmvqQfkRElrnlpQQCGlrapqrcdldvesggrdry,

comprising at least 1, 2, 3, 4, 5, 6, 7, 8, 9, 10, 11, 12, 13, 14, 15, or 16 substitutions at amino acid residues H29, Q32, R33, 134, G36, D53, Q55, Q56, C59, Q94, R97, E98, Q105, Q106, C107, G108, or a combination thereof. In some embodiments, the mutation is a non-conservative mutation, such as a charge-reversal mutation. In some embodiments, mutations of Gln, Glu, and/or Arg residues are preferred. Therefore, in some embodiments, the mutant Ara h 6 antigen comprises the amino acid sequence:
MRRERGRQGDSSSCERQVDRVNLKPCEQhlMqX₁iMgEQEQYDSYDIRSTRSSdQqX₂R CcDELNEMENTQGCMCEALQQIMENQCDRLQDRQMVQqFKrX₃LMSLPqX₄cnFRAPQR CDLDVSGGRC (SEQ ID NO:6), wherein X₁, X₂, X₃, and X₄ are not Arg, Gln, Glu, and Gln, respectively, such as wherein X₁, X₂, X₃, and X₄ are non-conservative substitutions of and Arg, Gln, Glu, and Gln, respectively. For example, in some embodiments, the mutant Ara h 6 antigen comprises the amino acid sequence:

(SEQ ID NO: 14)
MRRERGRQGDSSSCERQVDRVNLKPCEQhIMqEiMgEQEQY
DSYDIRSTRSSdQqARCcDELNEMENTQGCMCEALQQIMEN
QCDRLQDRQMVQqFKrRLMSLPqAcnFRAPQRCDLDVSGGR
C.

In some embodiments, the Ara h 2 wild type protein has the amino acid sequence:

(SEQ ID NO: 2)
RQQWELQGDRRCQSQLERANLRPCEQHLMQKIQRDEDSYER
DPYSPSQDPYSPSPYDRRGAGSSQHQERCCNELNEFENNQR
CMCEALQQIMENQSDRLQGRQQEQQFKRELRNLPQQCGLRA
PQRCDLDVESGGRDRY.

In some embodiments, an alternate Ara h 2 wild type protein has the amino acid sequence:

(SEQ ID NO: 20)
RQQWELQGDRRCQSQLERANLRPCEQHLMQKIQRDEDSYGR
DPYSPSQDPYSPSQDPDRRDPYSPSPYDRRGAGSSQHQERC
CNELNEFENNQRCMCEALQQIMENQSDRLQGRQQEQQFKRE
LRNLPQQCGLRAPQRCDLEVESGGRDRY.

In some embodiments, allergy-inducing antibodies bind a first set of contact residues in Ara h 2 (referred to herein as Ara h 2 Site A), as identified by the bold/uppercase residues in the amino acid sequence:

(SEQ ID NO: 2)
rqqwelqgdrrcqsqleranlrpceqhlmqkiqrdedsyerdp
yspsqdpyspspydrrgagssqhqerccnelnefeNNQrcmce
alqqimenqsdrlqgrqqeqqfkrelRnlpqqcglRAPQRcDl
DVEsggrdry

or
rqqwelqgdrrcqsqleranlrpceqhlmqkiqrdedsygrdpyspsqdpyspsqdpdrrdpyspspydrrgagssqhq erccnelnefeNNQrcmcealqqimenqsdrlqgrqqeqqfkrelRnlpqqcglRAPQRcDIEVesggrdry (SEQ ID NO:20). In some embodiments, any of these bold/uppercase residues can be mutated to disrupt binding by allergy-inducing antibodies. Therefore, in some embodiments, the mutant Ara h 2 antigen comprises the amino acid sequence:
rqqwelqgdrrcqsqleranlrpceqhlmqkiqrdedsyerdpyspsqdpyspspydrrgagssqhqerccnelnefeN NQrcmcealqqimenqsdrlqgrqqeqqfkrelRnlpqqcglRAPQRcDIDVEsggrdry (SEQ ID NO:2), comprising at least 1, 2, 3, 4, 5, 6, 7, 8, 9, 10, 11, 12, or 13 substitutions at amino acid residues N79, N80, Q81, R113, R122, A124, P124, Q125, R126, D128, D130, V131, E132, or a combination thereof. Therefore, in some embodiments, the mutant Ara h 2 antigen comprises the amino acid sequence:
rqqwelqgdrrcqsqleranlrpceqhlmqkiqrdedsygrdpyspsqdpyspsqdpdrrdpyspspydrrgagssqhq erccnelnefeNNQrcmcealqqimenqsdrlqgrqqeqqfkrelRnlpqqcglRAPQRcDIEVesggrdry (SEQ ID NO:20), comprising at least 1, 2, 3, 4, 5, 6, 7, 8, 9, 10, 11, 12, or 13 substitutions at amino acid residues N91, N92, Q93, R125, R134, A135, P136, Q137, R138, D140, E142, V143, or a combination thereof. In some embodiments, the mutation is a non-conservative mutation, such as a charge-reversal mutation. In some embodiments, the mutation is a non-conservative mutation, such as a charge-reversal mutation. In some embodiments, mutations of Gln, Glu, and/or Arg residues are preferred. Therefore, in some embodiments, the mutant Ara h 2 antigen comprises the amino acid sequence:
RQQWELQGDRRCQSQLERANLRPCEQHLMQKIQRDEDSYERDPYSPSQDPYSPSPY DRRGAGSSQHQERCCNELNEFEnnqRCMCEALQQIMENQSDRLQGRQQEQQFKRELr NLPQQCGLrapX₁X₂CdLdveSGGRDRY (SEQ ID NO:7), wherein X₁ and X₂ are not Gln and Arg, respectively, such as wherein X₁ and X₂ are non-conservative substitutions of Gln and Arg, respectively. For example, in some embodiments, the mutant Ara h 2 antigen comprises the amino acid sequence:

(SEQ ID NO: 15)
RQQWELQGDRRCQSQLERANLRPCEQHLMQKIQRDEDSYER
DPYSPSQDPYSPSPYDRRGAGSSQHQERCCNELNEFEnnqR
CMCEALQQIMENQSDRLQGRQQEQQFKRELr
NLPQQCGLrapAACdLdveSGGRDRY.

In some embodiments, allergy-inducing antibodies bind a second set of contact residues in Ara h 2 (referred to herein as Ara h 2 Site B), as identified by the bold/uppercase residues in the amino acid sequence:
rqqwelqgdrrcqsqleranlrpceqhlmqkiqrdedsyerDPYSPSqdpyspspydrrgagssqhqerccnelnefe nnqrcmcealqqimenqsdrlQGRQqeqqfkrelrnlpqqcglrapqrcdldvesggrdry (SEQ ID NO:2) or
rqqwelqgdrrcqsqleranlrpceqhlmqkiqrdedsygrDPYSPSqdpyspsqdpdrrdpyspspydrrgagssq hqerccnelnefennqrcmcealqqimenqsdrlQGRQqeqqfkrelrnlpqqcglrapqrcdlevesggrdry (SEQ ID NO:20). In some embodiments, any of these bold/uppercase residues can be mutated to disrupt binding by allergy-inducing antibodies. Therefore, in some embodiments, the mutant Ara h 6 antigen comprises the amino acid sequence:
rqqwelqgdrrcqsqleranlrpceqhlmqkiqrdedsyerDPYSPSqdpyspspydrrgagssqhqerccnelnefe nnqrcmcealqqimenqsdrlQGRQqeqqfkrelrnlpqqcglrapqrcdldvesggrdry (SEQ ID NO:2), comprising at least 1, 2, 3, 4, 5, 6, 7, 8, 9, 10, 11, or 12 substitutions at amino acid residues Y39, E40, R41, D42, P43, Y44, S45, P42, Q100, G101, R102, Q103, or a combination thereof. Therefore, in some embodiments, the mutant Ara h 6 antigen comprises the amino acid sequence:
rqqwelqgdrrcqsqleranlrpceqhlmqkiqrdedsygrDPYSPSqdpyspsqdpdrRDPyspspydrrgagss qhqerccnelnefennqrcmcealqqimenqsdrlQGRQqeqqfkrelrnlpqqcglrapqrcdlevesggrdry (SEQ ID NO:20), comprising at least 1, 2, 3, 4, 5, 6, 7, 8, 9, 10, 11, or 12 substitutions at amino acid residues Y39, G40, R41, D42, P43, Y44, S45, P42, R60, D61, P62, Q112, G113, R114, Q115, or a combination thereof. In some embodiments, the mutation is a non-conservative mutation, such as a charge-reversal mutation. In some embodiments, mutations of Gln, Glu, and/or Arg residues are preferred. Therefore, in some embodiments, the mutant Ara h 2 antigen comprises the amino acid sequence:
RQQWELQGDRRCQSQLERANLRPCEQHLMQKIQRDEDSyerdpyspSQDPYSPSPYDR RGAGSSQHQERCCNELNEFENNQRCMCEALQQIMENQSDRLqgX₁X₂QEQQFKRELR NLPQQCGLRAPQRCDLDVESGGRDRY (SEQ ID NO:8), wherein X₁ and X₂ are not Arg and Gln, respectively, such as wherein X₁ and X₂ are non-conservative substitutions of Arg and Gln, respectively. For example, in some embodiments, the mutant Ara h 2 antigen comprises the amino acid sequence:

(SEQ ID NO: 16)
RQQWELQGDRRCQSQLERANLRPCEQHLMQKIQRDEDSyer
dpyspSQDPYSPSPYDRRGAGSSQHQERCCNELNEFENNQR
CMCEALQQIMENQSDRLqgAAQEQQFKRELRNLPQQCGLRA
PQRCDLDVESGGRDRY.

As disclosed herein, site B involves a long loop that can be deleted and replaced with a short linker (e.g. GSG). Therefore, in some embodiments, the mutant Ara h 2 antigen comprises a deletion of D33-H66 in SEQ ID NO:2 or D33-H78 in SEQ ID NO:20. Therefore, in some embodiments, the mutant Ara h 2 antigen comprises the amino acid sequence:
rqqwelqgdrrcqsqleranlrpceqhlmqkiq(GSG)qerccnelnefennqrcmcealqqimenqsdrlQGRQqe qqfkrelrnlpqqcglrapqrcdldvesggrdry (SEQ ID NO:22). Therefore, in some embodiments, the mutant Ara h 2 antigen comprises the amino acid sequence:

(SEQ ID NO: 23)
rqqwelqgdrrcqsqleranlrpceqhlmqkiq(GSG)
qerccnelnefennqrcmcealqqimenqsdrlQGRQ
qeqqfkrelrnlpqqcglrapqrcdlevesggrdry

In some embodiments, allergy-inducing antibodies bind a third set of contact residues in Ara h 2 (referred to herein as Ara h 2 Site C), as identified by the bold/uppercase residues in the amino acid sequence:

(SEQ ID NO: 2)
rqqwelqgdrrcqsqleranlrpceqhlmqkiQrdedsyerd
pyspsqdpyspspydrrgagssqhqerccnelnefennqrcm
cealqqimenqSDrLQGRQQEQQfKrelrnlpqqcglrapqr
cdIDVeSGGRdry.

or rqqwelqgdrrcqsqleranlrpceqhlmqkiQrdedsygrdpyspsqdpyspsqdpdrrdpyspspydrrgagssqh qerccnelnefennqrcmcealqqimenqSDrLQGRQQEQQfKrelrnlpqqcglrapqrcdIEVeSGGRdry (SEQ ID NO:20). In some embodiments, any of these bold/uppercase residues can be mutated to disrupt binding by allergy-inducing antibodies. Therefore, in some embodiments, the mutant Ara h 2 antigen comprises the amino acid sequence:
rqqwelqgdrrcqsqleranlrpceqhlmqkiQrdedsyerdpyspsqdpyspspydrrgagssqhqerccnelnefen nqrcmcealqqimenqSDrLQGRQQEQQfKrelrnlpqqcglrapqrcdlDVeSGGRdry (SEQ ID NO:2), comprising at least 1, 2, 3, 4, 5, 6, 7, 8, 9, 10, 11, 12, 13, 14, 15, 16, 17, 18, or 19 substitutions at amino acid residues Q33, S96, D97, L98, Q100, G101, R102, Q103, Q104, E105, Q106, Q107, K109, D130, V131, S133, G134, G135, R136, ora combination thereof. Therefore, in some embodiments, the mutant Ara h 2 antigen comprises the amino acid sequence:
rqqwelqgdrrcqsqleranlrpceqhlmqkiQrdedsygrdpyspsqdpyspsqdpdrrdpyspspydrrgagssqh qerccnelnefennqrcmcealqqimenqSDrLQGRQQEQQfKrelrnlpqqcglrapqrcdlEVeSGGRdry (SEQ ID NO:20), comprising at least 1, 2, 3, 4, 5, 6, 7, 8, 9, 10, 11, 12, 13, 14, 15, 16, 17, 18, or 19 substitutions at amino acid residues Q33, S108, D109, L111, Q112, G113, R114, Q115, Q116, E117, Q118, Q119, K121, D142, V143, S145, G146, G147, R148, or a combination thereof. In some embodiments, the mutation is a non-conservative mutation, such as a charge-reversal mutation. In some embodiments, mutations of Gln, Glu, and/or Arg residues are preferred. Therefore, in some embodiments, the mutant Ara h 2 antigen comprises the amino acid sequence:
RQQWELQGDRRCQSQLERANLRPCEQHLMQKIqRDEDSYERDPYSPSQDPYSPSPY DRRGAGSSQHQERCCNELNEFENNQRCMCEALQQIMENQsdRlqgX₁X₂qeqqFkRELRN LPQQCGLRAPQRCDLdvEsggrDRY (SEQ ID NO:9), wherein X₁ and X₂ are not Arg and Gln, respectively, such as wherein X₁ and X₂ are non-conservative substitutions of Arg and Gln, respectively. For example, in some embodiments, the mutant Ara h 2 antigen comprises the amino acid sequence:

(SEQ ID NO: 17)
RQQWELQGDRRCQSQLERANLRPCEQHLMQKIqRDEDSYERDPYSP
SQDPYSPSPYDRRGAGSSQHQERCCNELNEFENNQRCMCEALQQIM
ENQsdRIqgAAqeqqFkRELRNLPQQCGLRAPQRCDLdvEsggrDRY.

In some embodiments, allergy-inducing antibodies bind a third set of contact residues in Ara h 2 (referred to herein as Ara h 2 Site D), as identified by the bold/uppercase residues in the amino acid sequence:

(SEQ ID NO: 2)
rqqwelqgdrrcqsqleranlrpceqHlmQKIqRdedsyerd
pyspsqdpyspspydrrgagssqhQErcCnelnefennqrcm
cealqqimenqsdrlqgrqqeqQfkRElrnlpQQCGlrapqr
cdldvesggrdry

or
rqqwelqgdrrcqsqleranlrpceqHlmQKlqRdedsygrdpyspsqdpyspsqdpdrrdpyspspydrrgagssq hQErcCnelnefennqrcmcealqqimenqsdrlqgrqqeqQfkRElrnlpQQCGlrapqrcdlevesggrdry (SEQ ID NO:20). In some embodiments, any of these bold/uppercase residues can be mutated to disrupt binding by allergy-inducing antibodies. Therefore, in some embodiments, the mutant Ara h 2 antigen comprises the amino acid sequence:

(SEQ ID NO: 2)
rqqwelqgdrrcqsqleranlrpceqHlmQKlqRdedsyerd
pyspsqdpyspspydrrgagssqhQErcCnelnefennqrcm
cealqqimenqsdrlqgrqqeqQfkRElrnlpQQCGlrapqr
cdldvesggrdry,

comprising at least 1, 2, 3, 4, 5, 6, 7, 8, 9, 10, 11, 12, 13, 14, or 15 substitutions at amino acid residues H27, Q30, K31, 132, R34, Q67, E68, C71, Q107, R110, E111, Q117, Q118, C119, G120, or a combination thereof. Therefore, in some embodiments, the mutant Ara h 2 antigen comprises the amino acid sequence:
rqqwelqgdrrcqsqleranlrpceqHlmQKlqRdedsygrdpyspsqdpyspsqdpdrrdpyspspydrrgagssq hQErcCnelnefennqrcmcealqqimenqsdrlqgrqqeqQfkRElrnlpQQCGlrapqrcdlevesggrdry (SEQ ID NO:20), comprising at least 1, 2, 3, 4, 5, 6, 7, 8, 9, 10, 11, 12, 13, 14, or 15 substitutions at amino acid residues H27, Q30, K31, 132, R34, Q79, E80, C83, Q119, R122, E123, Q129, Q130, C131, G132, or a combination thereof. In some embodiments, the mutation is a non-conservative mutation, such as a charge-reversal mutation. In some embodiments, mutations of Gln, Glu, and/or Arg residues are preferred. Therefore, in some embodiments, the mutant Ara h 2 antigen comprises the amino acid sequence:
RQQWELQGDRRCQSQLERANLRPCEQhLMqkiQrDEDSYERDPYSPSQDPYSPSPYD RRGAGSSQHqXRCcNELNEFENNQRCMCEALQQIMENQSDRLQGRQQEQqFKrX₂LR NLPqqcgLRAPQRCDLDVESGGRDRY(SEQ ID NO:10), wherein X₁ and X₂ are not Glu and Gln, respectively, such as wherein X₁ and X₂ are non-conservative and/or charge reversal substitutions of, such as wherein X₁ and X₂ are non-conservative substitutions of Glu and Gln, respectively. For example, in some embodiments, the mutant Ara h 2 antigen comprises the amino acid sequence:

(SEQ ID NO: 18)
RQQWELQGDRRCQSQLERANLRPCEQhLMqkiQrDEDSYERD
PYSPSQDPYSPSPYDRRGAGSSQHqARCcNELNEFENNQRCM
CEALQQIMENQSDRLQGRQQEQqFKrALRNLPqqcgLRAPQR
CDLDVESGGRDRY.

Also disclosed are polynucleotides encoding these polypeptide antigens. For example, these polynucleotides can be operably linked to an expression control sequence and incorporated into an expression vector. In some embodiments, the vector is a viral vector for administration to a subject.

### Vaccine Compositions

Disclosed herein are vaccine compositions containing the disclosed mutant peanut antigens for use in desensitizing or tolerizing a subject having a peanut allergy.

In some embodiments, disclosed mutant peanut antigens are present in a parental formulation in a compatible carrier. Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof.

The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

In some embodiments, the disclosed mutant peanut antigens are present in an oral formulation in compatible carrier. Suitable carriers mask the mutant peanut antigens from the mouth and upper gastrointestinal (GI) tract and reduce or prevent local itching/swelling reactions in these regions during administration. For example, a carrier may contain one or more lipid, polysaccaride or protein constituents. The carrier may be a food product, for example a dairy or dairy substitute product, such as yogurt, milkshake or chocolate, or another food product with similar properties. Dairy substitute products may include soy-based products.

In some embodiments, the composition for administration may be a food product which has been supplemented with mutant peanut antigens. The composition may be any food product which can be produced with a discrete dose of mutant peanut antigens, e.g. chocolate, yoghurt, confectionery (e.g. sweets and jellies) or beverages. In some embodiments, the composition may be a cooked or baked food product, such as a biscuit or cake. The mutant peanut antigens may be added at any stage of the production of the food product. The food product may be supplemented with flavorings to mask the taste of the mutant peanut antigens. Suitable food flavorings are well-known in the art and include sugar, mint, vanilla and orange essence. The food product may be supplemented with preservatives, stabilizing agents, fillers, colorings and sweeteners in accordance with standard food production techniques.

In other embodiments, the composition for administration may be an oral delivery vehicle such as a capsule, cachet or tablet, each of which contains a predetermined amount of peanut protein to provide the correct incremental dose to the patient. Oral delivery vehicles may be useful, for example, in avoiding contact between the peanut protein and the mouth and upper gastrointestinal tract. Suitable carriers, binders, fillers or diluents lubricants and preservatives for use in oral delivery vehicle are well known in the art.

In some embodiments, the composition for administration may further comprise other components, for example, anti-allergy drugs, such as antihistamines, steroids, bronchodilators, leukotriene stabilizers and mast cell stabilizers. Suitable anti-allergy drugs are well known in the art. This may be useful in reducing allergic inflammation and increasing tolerance of the peanut protein.

As described below, compositions, such as food products, for use as described herein may be formulated in unit dose formulations, which contain a defined amount of mutant antigens.

Anti-peanut IgE (such as those described herein) and/or anti-peanut IgG can be a surrogate marker for efficacy of the treatment. Typically, the level of anti-peanut IgG rises and anti-peanut IgE drops to low levels during the treatment described herein. Therefore, the disclosed methods can further involve assaying a sample from the subject for the presence of IgE and/or IgG antibodies that specifically bind the disclosed mutant peanut antigens. The method can also involve assaying a sample from the subject for the presence of IgE and/or IgG antibodies that specifically bind wildtype peanut antigens.

Following the treatment, levels of anti-peanut IgE may be reduced or abolished. Weekly administration of the maximum incremental dose may continue until the anti-peanut IgE level in the patient serum is minimized and the patient is no longer reactive to peanut protein. For example, anti-peanut IgE levels may be reduced to zero, substantially zero, or very low levels. In some embodiments, the level of IgE reactive to specific allergens, such as Ara h 2 or Ara h 2, may be measured in the serum of the patient.

Methods described herein may be useful in the treatment of peanut allergy. For example, the sensitivity of the patient to peanut protein may be reduced or abolished following the treatment. For example, the maximum oral dose of peanut allergen which is tolerated by a patient without the onset of allergic symptoms, or the median maximum oral dose of peanut allergen which is tolerated by a population of patients, may be increased by at least 50 fold, at least 500 fold, at least 750 fold or at least 1000 fold after the treatment relative to before the treatment.

Also disclosed are individual unit dose formulations comprising mutant peanut antigens, wherein the amount of mutant peanut antigens in each unit dosage formulation in the set is identical and is selected from 0.5 mg, 1 mg, 2 mg, 5 mg, 12.5 mg, 25 mg, 50 mg, 100 mg, 200 mg, 400 mg, and 800 mg. Also disclosed is a kit containing 5 or more, 10 or more, 14 or more, 15 or more, 20 or more, 25 or more, 30 or more, or 35 or more unit dose formulations. In some embodiments, the set may comprise unit dose formulations sufficient for two, three, or four weeks administration of the incremental dose. The unit dose formulations may be separately packaged in sealed units, for example to protect the contents from the external environment. The unit dose formulations may be packaged in separate sealed containers, for example wrappers, pouches, bags, cartons, capsules, sachets, vials or tubes.

### Assay Methods

Human IgE antibodies from secreting cells lines were isolated from peanut allergic patients that specifically bind peanut allergy antigens Ara h 2 and Ara h 6. Disclosed herein are the antigen sites where these antibodies bind. Table 1 shows EC₅₀ of Ara h 2- and/or h 6-specific IgE mAbs against the identified antigenic sites.

TABLE 1
EC₅₀ Ara h 2 and/or h6 specific IgE mAbs

| Human IgE | Antigenic | Ara h 2 | Ara h 6 |
|---|---|---|---|
| mAbs | Site | EC₅₀ (ng/mL) | EC₅₀ (ng/mL) |
| 5C5 | Site A | 2.7 | 10.8 |
| 32G9 | Site A | 10.9 | 14.3 |
| 16A8 | Site A | 5.8 | 17.6 |
| 29B3 | Site A | 15.5 | 16.9 |
| 24F10 | Site A | 4.3 | 4.0 |
| 14B3 | Site A | 41.6 | 9.0 |
| 15A7 | Site A | 10.7 | 10.0 |
| 2F12 | Site A | 15.5 | 25.5 |
| 5D7 | Site A | 9.3 | 11.7 |
| 16G12 | Site A/D | 25.6 | 40.7 |
| 9H11 | Site A/E | 3.9 | 1.3 |
| 15A4 | Site B | 17.6 | > |
| 26C3 | Site B | 6.5 | > |
| 38B7 | Site B | 11.6 | > |
| 15B8 | Site B | 6.9 | 163.2 |
| 13D9 | Site B | 21.3 | 42.0 |
| 2C9 | Site B | > | 17.4 |
| 7B6 | Site B | > | 30 |
| 11F10 | Site B/C | 14.1 | 15.4 |
| 1H9 | Site C | > | 12.3 |
| 22F2 | Site C | > | 5.5 |
| 15C2 | Site C | > | 31.5 |
| 26A5 | Site C | > | 10.1 |
| 6E10 | Site C | > | 9.1 |
| 8F3 | Site D | > | 3.8 |
| 1A8 | Site D | > | 5.0 |
| 20M10 | Site D/E | > | 5.3 |
| 21C10 | Site E | > | 3.2 |
| 3F9 | Site E | > | 1.7 |
| 20G11 | Site F | 173.5 | 22.7 |

All IgE mAbs. > denotes an EC₅₀ is greater than 250 ng/mL.

The disclosed mutant antigens can therefore be used to detect the presence of antibodies in a subject capable of binding these antigens. This includes both the naturally occurring Ara h 2 and Ara h 6 proteins as well as the mutant antigens. These antigens can be used to monitor the disclosed therapeutic methods to determine if levels of IgE antibodies capable of binding the disclosed allergy antigen sites are decreasing as a result of the therapy. For example, the disclosed antigens can be used in an immunoassay to capture antibodies capable of binding the disclosed antigens.

Secondary antibodies capable of binding IgE or IgG can in some embodiments be used to determine whether there is a decrease in IgE antibodies and an associated increase in IgG antibodies that bind the peanut antigens as a result of the therapy. In some embodiments, the disclosed antigens could be used to assay blood or tissue before transfusion or transplant. In some embodiments, the disclosed antigens could be used as scientific reagents for the study or allergy or biology related to allergy in experimental systems.

### Therapeutic Methods

Disclosed herein are methods for desensitizing or tolerizing a subject with a peanut allergy to the peanut allergens. Therefore, disclosed herein are compositions, products, vectors and formulations for use in preventing or treating allergy to peanuts by tolerization. The invention also provides a method of tolerizing or desensitizing a peanut allergic individual that involves administering, either singly or in combination the disclosed mutant antigens as described above.

The method involves administering an effective amount of a mutant peanut antigen as provided herein or a vaccine composition as provided herein to a mammal e.g. a human. For example, the method can involve administering an effective amount of a mutant peanut antigen according to any one of SEQ ID NOs:3-18, or a vaccine composition comprising such an allergen.

The individual to be treated or provided with the disclosed compositions or formulations is preferably human. It will be appreciated that the individual to be treated may be known to be sensitized to the allergens, at risk of being sensitized or suspected of being sensitized. The individual can be tested for sensitization using techniques well known in the art and as described herein. Alternatively, the individual may have a family history of allergy to peanuts. It may not be necessary to test an individual for sensitization to peanuts because the individual may display symptoms of allergy when exposed to peanuts. Symptoms of allergy can include itchy eyes, runny nose, breathing difficulties, red itchy skin or rash.

The individual to be treated may be of any age. However, preferably, the individual may be in the age group of 1 to 90, 5 to 60, 10 to 40, or more preferably 18 to 35.

Administration of the disclosed mutant peanut antigens may be by any suitable method as described herein. Suitable amounts of the peptide may be determined empirically, but typically are in the range given below. A single administration of each peptide may be sufficient to have a beneficial effect for the patient, but it will be appreciated that it may be beneficial if the peptide is administered more than once, in which case typical administration regimes may be, for example, once or twice a week for 2-4 weeks every 6 months, or once a day for a week every four to six months.

Dosages for administration will depend upon a number of factors including the nature of the composition, the route of administration and the schedule and timing of the administration regime. Suitable doses of a molecule of the invention may be in the order of up to 15 μg, up to 20 μg, up to 25 μg, up to 30 μg, up to 50 μg, up to 100 μg, up to 500 μg or more per administration. Suitable doses may be less than 15 μg, but at least 1 ng, or at least 2 ng, or at least 5 ng, or at least 50 ng, or least 100 ng, or at least 500 ng, or at least or at least 10 μg. For some molecules of the invention, the dose used may be higher, for example, up to 1 mg, up to 2 mg, up to 3 mg, up to 4 mg, up to 5 mg or higher. Such doses may be provided in a liquid formulation, at a concentration suitable to allow an appropriate volume for administration by the selected route.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

Human IgE antibodies secreting cell lines have been isolated from peanut allergic patients and co-crystal structures have been determined in which the peanut allergens Ara h 2 and Ara h 6 are bound to antibody fragments containing the IgE derived variable regions. These crystal structures reveal the molecular details of allergens Ara h 2 and Ara h 6 that are recognized by disease causing antibodies. In this application, the new antibody-allergen crystal structures were used to define the molecular epitopes recognized in disease, and to introduce mutations into Ara h 6 that prevent IgE binding. These mutant allergens are unique in that they do not exist in nature, and are commercially valuable. As disclosed herein, they can be used as the basis of a novel diagnostic and as the basis of a novel immunization strategy to treat peanut allergy.

### Ara h 6:16A8 Complex

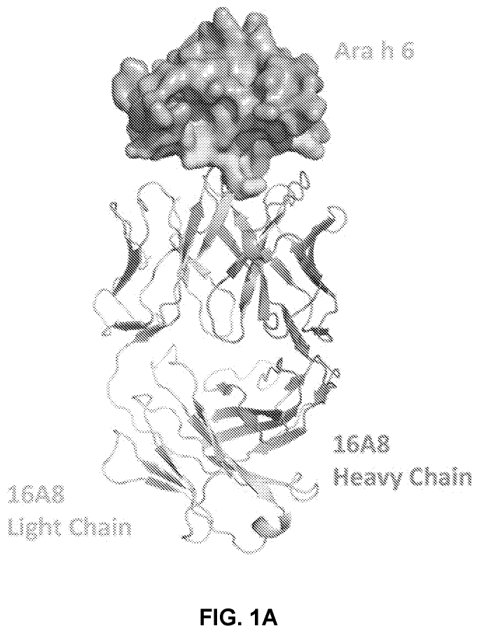

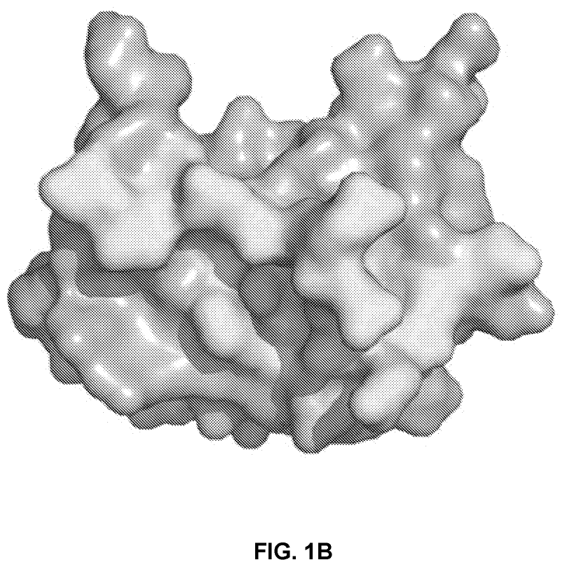

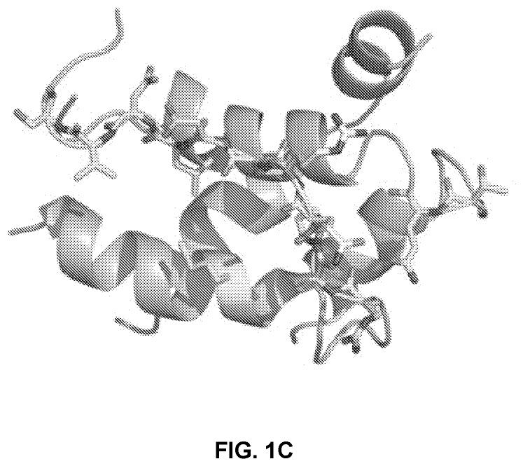

IgE 16A8 is a site A antibody. It binds the first-identified antigenic site on Ara h 2 and on Ara h 6. We have determined the structure of Ara h 6 bound to a Fab fragment containing the 16A8 variable region (FIG. 1). The interaction is approximately centered on residue 114 of Ara h 6 and involves residues 68, 69, 70, 101, and 110-120. These residues form a nearly contiguous patch (FIG. 1).

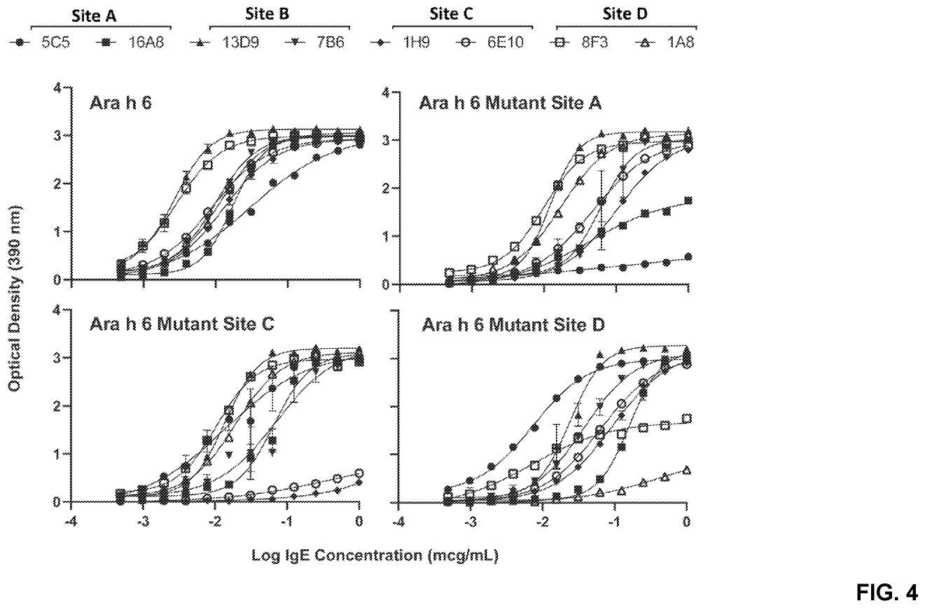

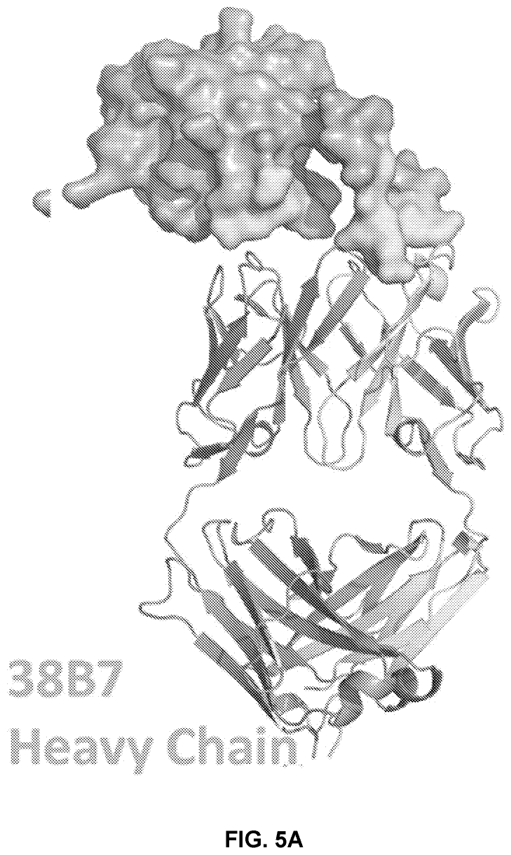

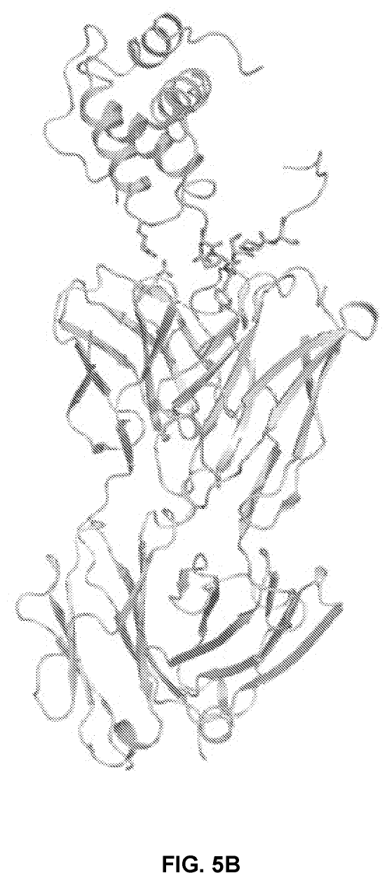

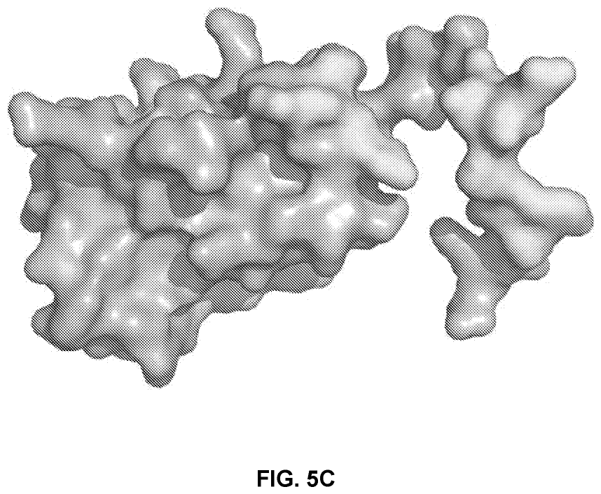

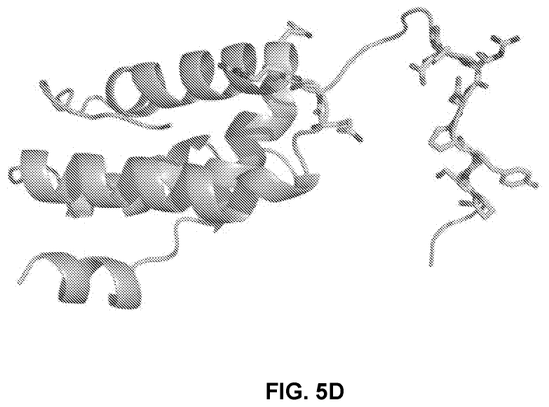

Analysis of the structure revealed Q113 and R114 of Ara h 6 to be critical for this interaction. R114 is recognized by three negatively charged residues, D91 from the L chain, D57 from the H chain, and E33 from the H chain. We show, FIG. 4, that mutation of R114 and Q113 disrupts binding to multiple site A binding IgE antibodies (16A8 and 5C5), but not to other anti Ara h 2/Ara h 6 antibodies that do not target site A (FIG. 4).

### Ara h 6:1H9 Complex

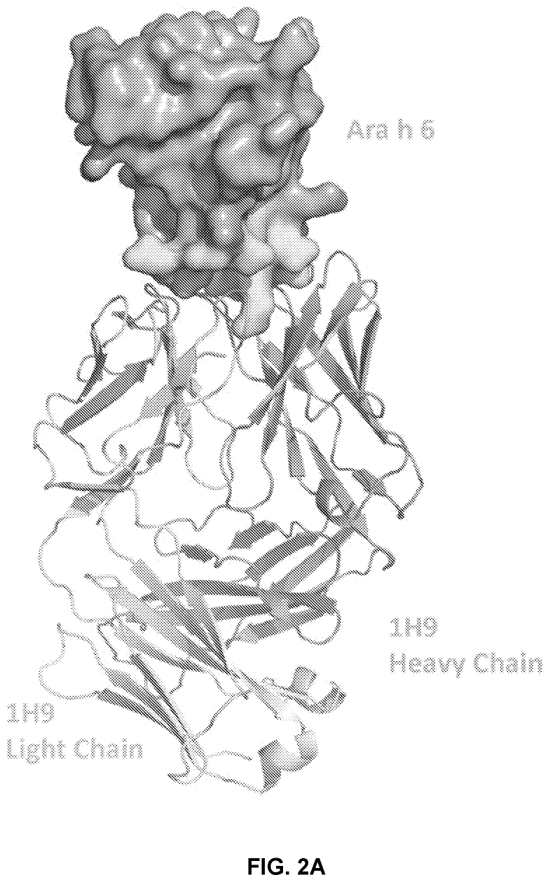

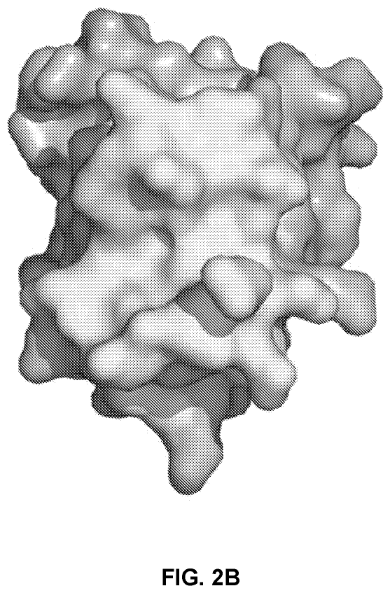

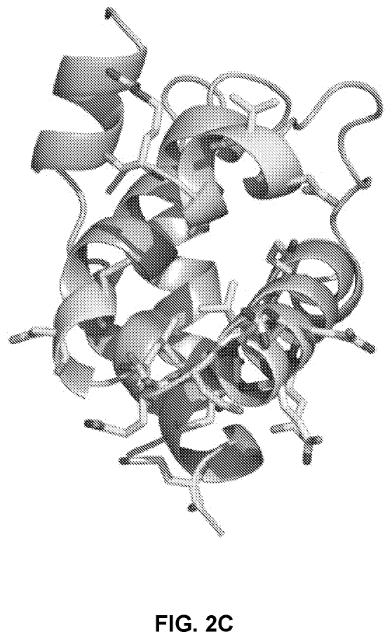

IgE 1H9 is a site C antibody. It binds the third-identified antigenic site on Ara h 2 and Ara h 6. The structure of Ara h 6 bound to both 1H9 and 8F3 was determined (described below). 1H9 binds across a surface made of the carboxy terminal portion of the 4^{th} alpha helix, the amino terminal portion of the 5^{th} alpha helix, and the carboxy terminal region of the 6^{th}, and final helix of Ara h 6 (FIG. 2). Residues on Ara h 6 recognized by 1H9 are 35, 84, 85, 87-95, 97, 118, 119, and 121-124 (FIG. 2).

Analysis of this interface indicated that R90 and Q91 on Ara h 6 were centrally located in the interface and recognized by a salt bridges and hydrogen bonds. A structure guided double mutant was introduced to Ara h 6, R90E and Q91A (FIG. 4). This mutant is defective in binding to multiple site C IgE (1H9 and 6E10), but is recognized by non-site C targeted anti Ara h 2/Ara h 6 antibodies (FIG. 4).

Ara h 6:8F3 complex, Site D

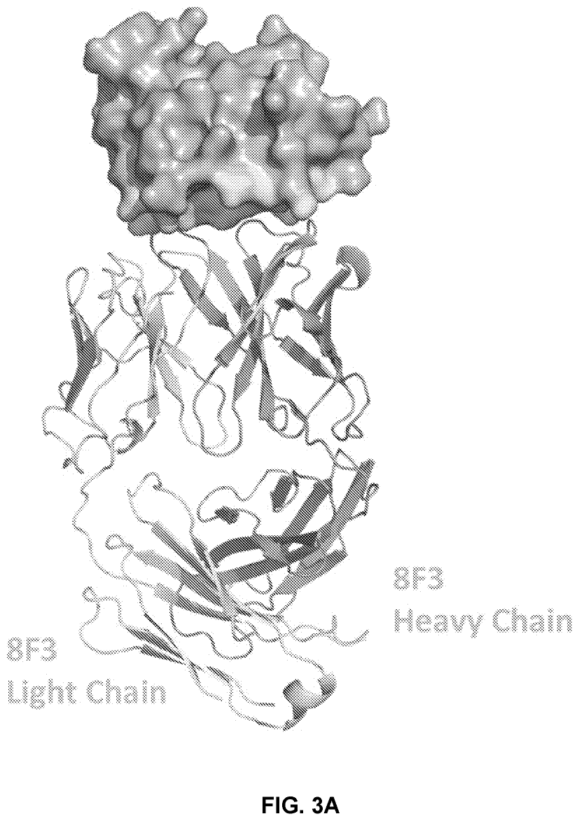

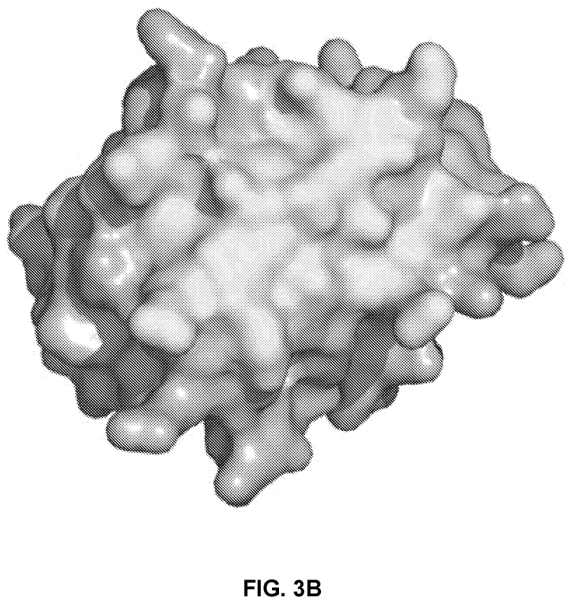

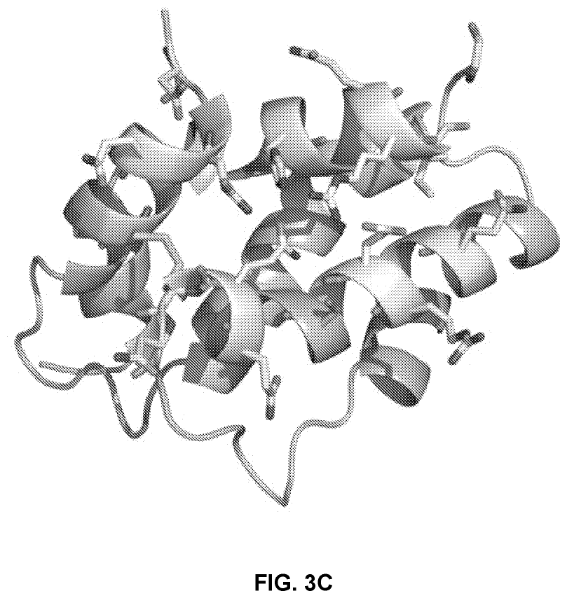

IgE 8F3 is a site D antibody. It binds the fourth-identified antigenic site on Ara h 2 and Ara h 6. 8F3 recognizes the side of the 5^{th} helix of Ara h 6 as well as the carboxy terminal ends of helix 2 and 3 (FIG. 3). Residues on Ara h 6 recognized by 1H9 are 18, 21, 22, 23, 25, 42, 44, 45, 48, 84, 87, 88, 91, 94, 95, 96, and 97. These residues form a contiguous patch (FIG. 3). Structural analysis revealed residues 22, 44, 88, and 95 as being important for the interaction. Mutation of these residues disrupts binding to 8F3, but not to antibodies that recognize sites A, B, or C (FIG. 4).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising a mutant peanut (Arachis hypogaea) Ara h 6 polypeptide in a pharmaceutically acceptable carrier, wherein the mutant Ara h 6 polypeptide has a reduced IgE response in a subject with a peanut allergy compared to wildtype Ara h 6, and
wherein the mutant Ara h 6 polypeptide has the amino acid sequence SEQ ID NO:1 with at least 1, 2, 3, 4, 5, 6, 7, 8, 9, 10, 11, 12, 13, or 14 substitutions at amino acid residues T68, Q69, M101, R110, A111, P112, Q113, R114, D104, D118, V119, S120, or a combination thereof.

2. (canceled)

3. The composition of claim 1, wherein the mutant Ara h 6 polypeptide has the amino acid sequence SEQ ID NO:3.

4. The composition of claim 3, wherein the mutant Ara h 6 polypeptide has the amino acid sequence SEQ ID NO:11.

5. The composition of claim 1, wherein the mutant Ara h 6 polypeptide has the amino acid sequence SEQ ID NO:1 with at least 1, 2, 3, 4, 5, or 6 substitutions at amino acid residues Y44, D45, Q88, D89, R90, Q91, or a combination thereof.

6. The composition of claim 5, wherein the mutant Ara h 6 polypeptide has the amino acid sequence SEQ ID NO:4.

7. The composition of claim 6, wherein the mutant Ara h 6 polypeptide has the amino acid sequence SEQ ID NO:12.

8. The composition of claim 1, wherein the mutant Ara h 6 polypeptide has the amino acid sequence SEQ ID NO:1 with at least 1, 2, 3, 4, 5, 6, 7, 8, 9, 10, 11, 12, 13, 14, 15, 16, 17, or 18 substitutions at amino acid residues M35, C84, D85, L87, Q88, D89, R90, Q91, M92, V93, Q94, Q95, K97, D118, V119, G121, G122, R123, C124, or a combination thereof.

9. The composition of claim 8, wherein the mutant Ara h 6 polypeptide has the amino acid sequence SEQ ID NO:5.

10. The composition of claim 9, wherein the mutant Ara h 6 polypeptide has the amino acid sequence SEQ ID NO:13.

11. The composition of claim 1, wherein the mutant Ara h 6 polypeptide has the amino acid sequence SEQ ID NO:1 with at least 1, 2, 3, 4, 5, 6, 7, 8, 9, 10, 11, 12, 13, 14, 15, or 16 substitutions at amino acid residues H29, Q32, R33, 134, G36, D53, Q55, Q56, C59, Q94, R97, E98, Q105, Q106, C107, N108, or a combination thereof.

12. The composition of claim 11, wherein the mutant Ara h 6 polypeptide has the amino acid sequence SEQ ID NO:6.

13. The composition of claim 13, wherein the mutant Ara h 6 polypeptide has the amino acid sequence SEQ ID NO:14.

14-25. (canceled)

26. A method for desensitizing or tolerizing a subject with a peanut allergy, comprising administering to the subject the composition of claim 1.

27. A method for monitoring progress for the method of claim 26, comprising assaying a sample from the subject for IgE and/or IgG antibodies that selectively bind one or more mutant peanut polypeptides, wherein a reduction in IgE antibodies and/or an increase in IgG antibodies that selectively bind the one or more mutant peanut polypeptides is an indication that the subject is being desensitized or tolerized to the peanut allergy

28. A composition comprising a mutant peanut (Arachis hypogaea) Ara h 2 polypeptide in a pharmaceutically acceptable carrier, wherein the mutant Ara h 2 polypeptide has a reduced IgE response in a subject with a peanut allergy compared to wildtype Ara h 2 polypeptide, and
wherein the mutant Ara h 2 polypeptide has the amino acid sequence SEQ ID NO:2 with at least 1, 2, 3, 4, 5, 6, 7, 8, 9, 10, 11, 12, or 13 substitutions at amino acid residues N79, N80, Q81, R113, R122, A123, P124, Q125, R126, D128, D130, V131, E132, or a combination thereof.


SEQUENCE LISTING (23 sequences)

SEQ ID NO: 1 - protein, 124 aa, Arachis hypogaea
MRRERGRQGDSSSCERQVDRVNLKPCEQHIMQRIMGEQEQYDSYDIRSTRSSDQQQRCCDELNEMENTQGCMCEALQQIMENQCDRLQDRQMVQQFKRELMSLPQQCNFRAPQRCDLDVSGGRC

SEQ ID NO: 2 - protein, 139 aa, Arachis hypogaea
RQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGRQQEQQFKRELRNLPQQCGLRAPQRCDLDVESGGRDRY

SEQ ID NO: 3 - protein, 124 aa, synthetic construct
  VARIANT 113: note = Xaa is not Gln
  VARIANT 114: note = Xaa is not Arg
MRRERGRQGDSSSCERQVDRVNLKPCEQHIMQRIMGEQEQYDSYDIRSTRSSDQQQRCCDELNEMENTQGCMCEALQQIMENQCDRLQDRQMVQQFKRELMSLPQQCNFRAPXXCDLDVSGGRC

SEQ ID NO: 4 - protein, 124 aa, synthetic construct
  VARIANT 90: note = Xaa is not Arg
  VARIANT 91: note = Xaa is not Gln
MRRERGRQGDSSSCERQVDRVNLKPCEQHIMQRIMGEQEQYDSYDIRSTRSSDQQQRCCDELNEMENTQGCMCEALQQIMENQCDRLQDXXMVQQFKRELMSLPQQCNFRAPQRCDLDVSGGRC

SEQ ID NO: 5 - protein, 124 aa, synthetic construct; identical to SEQ ID NO: 4
  VARIANT 90: note = Xaa is not Arg
  VARIANT 91: note = Xaa is not Gln

SEQ ID NO: 6 - protein, 124 aa, synthetic construct
  VARIANT 33: note = Xaa is not Arg
  VARIANT 56: note = Xaa is not Gln
  VARIANT 99: note = Xaa is not Glu
  VARIANT 106: note = Xaa is not Gln
MRRERGRQGDSSSCERQVDRVNLKPCEQHIMQXIMGEQEQYDSYDIRSTRSSDQQXRCCDELNEMENTQGCMCEALQQIMENQCDRLQDRQMVQQFKRXLMSLPQXCNFRAPQRCDLDVSGGRC

SEQ ID NO: 7 - protein, 139 aa, synthetic construct
  VARIANT 125: note = Xaa is not Gln
  VARIANT 126: note = Xaa is not Arg
RQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGRQQEQQFKRELRNLPQQCGLRAPXXCDLDVESGGRDRY

SEQ ID NO: 8 - protein, 139 aa, synthetic construct
  VARIANT 102: note = Xaa is not Arg
  VARIANT 103: note = Xaa is not Gln
RQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGXXQEQQFKRELRNLPQQCGLRAPQRCDLDVESGGRDRY

SEQ ID NO: 9 - protein, 139 aa, synthetic construct; identical to SEQ ID NO: 8
  VARIANT 102: note = Xaa is not Arg
  VARIANT 103: note = Xaa is not Gln

SEQ ID NO: 10 - protein, 139 aa, synthetic construct
  VARIANT 68: note = Xaa is not Glu
  VARIANT 111: note = Xaa is not Gln
RQQWELQGDRRCQSQLERANLRPCEQHLMQKIQRDEDSYERDPYSPSQDPYSPSPYDRRGAGSSQHQXRCCNELNEFENNQRCMCEALQQIMENQSDRLQGRQQEQQFKRXLRNLPQQCGLRAPQRCDLDVESGGRDRY

SEQ ID NO: 11 - protein, 248 aa, synthetic construct
MRRERGRQGDSSSCERQVDRVNLKPCEQHIMQRIMGEQEQYDSYDIRSTRSSDQQQRCCDELNEMENTQGCMCEALQQIMENQCDRLQDRQMVQQFKRELMSLPQQCNFRAPAECDLDVSGGRCMRRERGRQGDSSSCERQVDRVNLKPCEQHIMQRIMGEQEQYDSYDIRSTRSSDQQQRCCDELNEMENTQGCMCEALQQIMENQCDRLQDRQMVQQFKRELMSLPQQCNFRAPAACDLDVSGGRC

SEQ ID NO: 12 - protein, 112 aa, synthetic construct
SCERQVDRVNLKPCEQHIMQRIMGEQEQYDSYDIRSTRSSDQQQRCCDELNEMENTQGCMCEALQQIMENQCDRLQDAAMVQQFKRELMSLPQQCNFRAPQRCDLDVSGGRC

SEQ ID NO: 13 - protein, 124 aa, synthetic construct
MRRERGRQGDSSSCERQVDRVNLKPCEQHIMQRIMGEQEQYDSYDIRSTRSSDQQQRCCDELNEMENTQGCMCEALQQIMENQCDRLQDEAMVQQFKRELMSLPQQCNFRAPQRCDLDVSGGRC

SEQ ID NO: 14 - protein, 124 aa, synthetic construct
MRRERGRQGDSSSCERQVDRVNLKPCEQHIMQEIMGEQEQYDSYDIRSTRSSDQQARCCDELNEMENTQGCMCEALQQIMENQCDRLQDRQMVQQFKRRLMSLPQACNFRAPQRCDLDVSGGRC

SEQ ID NO: 15 - protein, 139 aa, synthetic construct
RQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGRQQEQQFKRELRNLPQQCGLRAPAACDLDVESGGRDRY

SEQ ID NO: 16 - protein, 139 aa, synthetic construct
RQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGAAQEQQFKRELRNLPQQCGLRAPQRCDLDVESGGRDRY

SEQ ID NO: 17 - protein, 139 aa, synthetic construct; identical to SEQ ID NO: 16

SEQ ID NO: 18 - protein, 139 aa, synthetic construct
RQQWELQGDRRCQSQLERANLRPCEQHLMQKIQRDEDSYERDPYSPSQDPYSPSPYDRRGAGSSQHQARCCNELNEFENNQRCMCEALQQIMENQSDRLQGRQQEQQFKRALRNLPQQCGLRAPQRCDLDVESGGRDRY

SEQ ID NO: 19 - protein, 127 aa, synthetic construct
MRRERGRQGDSSSCERQVDRVNLKPCEQHIMQRIMGEQEQYDSYDIRSTRSSDQQQRCCDELNEMENTQGCMCEALQQIMENQCDRLQDRQMVQQFKRELRNLPQQCGLRAPQRCDLDVESGGRDRY

SEQ ID NO: 20 - protein, 151 aa, synthetic construct
RQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 21 - protein, 105 aa, synthetic construct
MRRERGRQGDSSSCERQVDRVNLKPCEQHIMQRGSGQRCCDELNEMENTQGCMCEALQQIMENQCDRLQDRQMVQQFKRELMSLPQQCNFRAPQRCDLDVSGGRC

SEQ ID NO: 22 - protein, 109 aa, synthetic construct
RQQWELQGDRRCQSQLERANLRPCEQHLMQKIQGSGQERCCNELNEFENNQRCMCEALQQIMENQSDRLQGRQQEQQFKRELRNLPQQCGLRAPQRCDLDVESGGRDRY

SEQ ID NO: 23 - protein, 109 aa, synthetic construct
RQQWELQGDRRCQSQLERANLRPCEQHLMQKIQGSGQERCCNELNEFENNQRCMCEALQQIMENQSDRLQGRQQEQQFKRELRNLPQQCGLRAPQRCDLEVESGGRDRY